(19) United States
(12) Patent Application Publication
(10) Pub. No.: US 2026/0183091 A1
(43) Pub. Date: Jul. 2, 2026

(54) PREOPERATIVE PLANNING FOR INTRODUCING A DENTAL IMPLANT

(71) Applicant: ALBERT-LUDWIGS-UNIVERSITÄT FREIBURG, Freiburg (DE)

(72) Inventors: Marc METZGER, Kirchzarten (DE); Leonard BRANDENBURG, Freiburg (DE); Stefan SCHLAGER, Freiburg (DE)

(21) Appl. No.: 18/727,622

(22) PCT Filed: Dec. 29, 2022
(86) PCT No.: PCT/EP2022/088045

§ 371 Date: Jul. 9, 2024

(30) Foreign Application Priority Data
Jan. 10, 2022 (DE) 102022100438.4

(51) Int. Cl. A61C 13/00; A61B 34/10; A61C 1/08

(52) U.S. Cl. CPC A61C 13/0004; A61B 34/10; A61C 1/084; A61B 2034/102; A61B 2034/105

(57) ABSTRACT

The invention relates to a computer-implemented method, a system and a computer program product for supporting preoperative planning of placement of a dental implant in a patient's jaw. A dental axis representation, a dental crown representation and/or an implantation axis representation is generated using a provided digital 3D surface visualization, e.g. a digital plaster model, at least part of the existing dental crowns and/or the oral mucosa of the patient and statistical parametric 3D shape models (SSM). This is done based on the digital 3D surface visualization and the corresponding statistical parametric 3D shape model. In addition, the method, system and computer program product can also be used to generate a corresponding statistical parametric 3D shape model. For a plausibility check, the determined representations for the tooth axis, crown and/or implantation axis can be displayed in an additionally recorded 2D X-ray image, e.g. an orthopantomogram.

### TECHNICAL AREA

The invention relates to a computer-implemented method, a system and a computer program product for supporting a preoperative planning of a placement of a dental implant in a jaw of a patient.

### STATE OF THE ART

Implant-supported prostheses are the current gold standard in the treatment of partially toothed patients, and their use is steadily increasing. Supported by the ongoing development of digital workflows, surgical implant insertion, i.e. the placement of an implant in the jawbone, is becoming increasingly improved and more cost-effective.

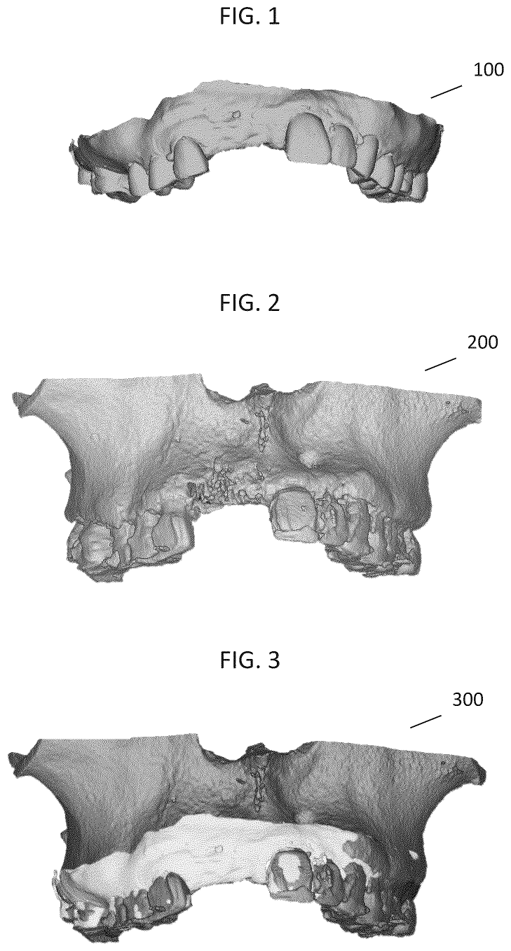

Currently, the surgical insertion of dental implants is planned on the basis of 3-dimensional surface models or digital surface representations of the jaws (digitized plaster models or intraoral scans), as shown in FIG. 1, and 3-dimensional cone-beam computed tomography (CBCT) images, as shown in FIG. 2.

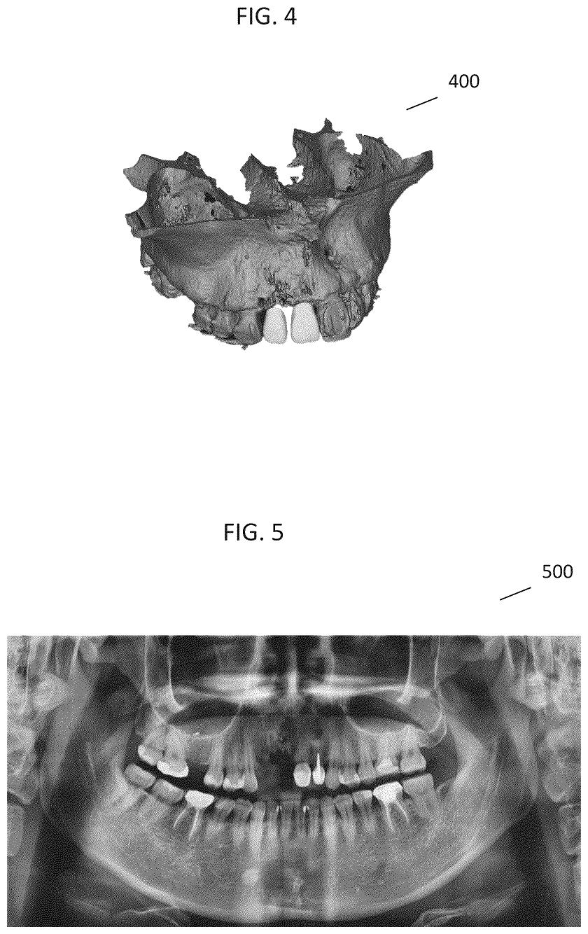

The surface model and CBCT image are then superimposed in virtual space, as shown in FIG. 3, for example, and the dental crown(s) to be replaced are virtually incorporated into the tooth space by a doctor/dentist (see FIG. 4, for example).

In addition, X-ray overview images (e.g. orthopantomograms, OPGs) are taken, as these, unlike CBCT images, make it possible to assess whether individual teeth are worth preserving (see e.g. FIG. 5).

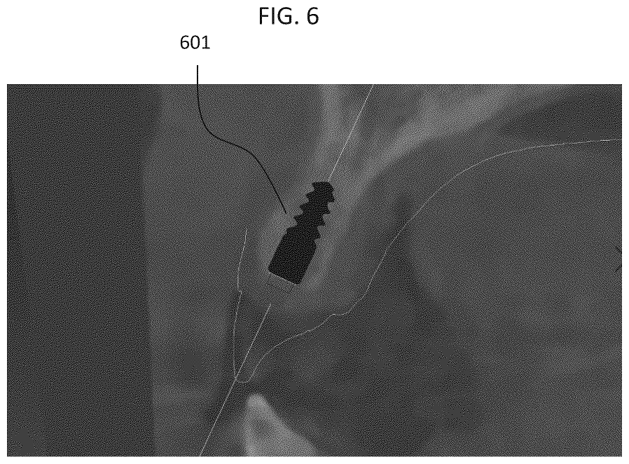

By examining the superimposed data set (FIG. 4), anatomical and prosthetic factors are taken into account to determine the position and alignment of the implant. The anatomy of the surrounding structures is assessed using the CBCT scan. Neighboring teeth and surrounding nerves must not run too close to the implant and the bone supply must be sufficient for firm anchoring of the implant. Finally, a suitable position and axis of an implant 601 are determined as shown in FIG. 6, taking into account the previously planned tooth replacement and the surrounding anatomical structures.

Finally, a drilling template can be produced using a 3D printing process, for example, to enable precise transfer of the planned implant position/axis during the operation.

The described workflow of the current state of the art is a complex process that requires trained personnel to operate the corresponding planning software. In addition, expensive software licenses are required to carry out the planning and generation of a drilling template. Both factors lead to high costs. Above all, however, the process is associated with considerable radiation exposure in a radiation-sensitive anatomical area. For example, the head and neck area with the sensory organs and parts of the central nervous system are regularly exposed to comparatively high doses of radiation during CBCT scans. Compared to conventional radiography, 3-dimensional imaging using CBCT applies around 23 times the radiation dose, i.e. around 530 μSv (cf. OPG image approx. 23 μSv).

US2018085201 A1 and a related scientific publication (“Model-Based Teeth Reconstruction”, Wu et al., DOI: http://dy.doi.org/10.1145/2980179.2980233) describe a method and system for reconstructing individual teeth or entire rows of teeth based on a statistical shape model generated using digitized plaster casts or plaster models. The focus of the approach is on creating realistic animations. However, the underlying 3D tooth models are designed with the artistic aim of achieving the best possible aesthetics. An exact calculation of anatomical covariances, such as the precise prediction of the course of the tooth root, is therefore not possible on the basis of this work. However, information about the tooth roots, the tooth axes and their anatomical covariation with the optically detectable tooth crowns that is as close to reality as possible is essential for implant planning if no three-dimensional cross-sectional imaging procedure is available.

In the article “Can we estimate root axis using a 3-dimensional tooth model via lingual-surface intraoral scanning?” (Lim et al., Am J Orthod Dentofacial Orthop. 2020 November; 158(5):e99-e109. doi: 10.1016/j.ajodo.2020.07.032), the authors discuss the estimation of the root axis using 3D tooth models. However, in the approaches discussed, a CBCT image is taken for each patient in order to create a 3D tooth model using segmentation and to overlay this with a new CBCT image during the course of treatment. The previously discussed problem of the associated radiation can therefore also not be avoided with this workflow.

The Ortho Insight 3D software (Motion View LLC, Chattanooga, Tennessee, USA) attempts to predict the shape of tooth roots using standard tooth models. However, studies investigating the accuracy of this approach report large deviations of 9.2-22.5° and 7.95-15° between the actual and estimated root axis on average (Magkavali-Trikka et al, “Estimation of root inclination of anterior teeth from virtual study models: accuracy of a commercial software”, Prog Orthod. 2019; 20. doi:10.1186/s40510-019-0298-5; Dastoori et al, “Anterior teeth root inclination prediction derived from digital models: A comparative study of plaster study casts and CBCT images”, J Clin Exp Dent. 2018; 10(11):e1069-e1074. doi:10.4317/jced.55180).

In WO 2021/046147 A1, it is proposed to determine a 3D shape of a restorative dental object by means of a machine learning system and a 3D representation of a 3D scan of at least part of a patient's dentition. However, automatic surgery planning is excluded.

WO 2020/227661 A1 discusses generating a three-dimensional anatomical model based on medical image data and adapting a statistical shape model to it. Based on the adapted statistical shape model, quantitative measurements are performed to classify a defect related to the patient's anatomy.

### Task and Solution of the Invention

Against this background, the aim of the invention is to improve implant planning while simultaneously reducing radiation exposure.

The invention solves this task by means of a computer-implemented method according to claim 1, a system according to claim 12 and a computer program product according to claim 22.

One aspect of the invention relates to a computer-implemented method for supporting preoperative planning of placement of a dental implant in a patient's jaw. In a first step, a digital 3D surface visualization of at least a part of the existing dental crowns and/or the oral mucosa of the patient is provided. Such a digital 3D surface visualization can, for example, be a surface scan that was or is generated, for example, directly by means of an intra-oral scanner or by means of a plaster model scanned in 3D by a laboratory scanner. In a second step, a statistical parametric 3D shape model is provided, which is suitable for generating representations of the tooth axes as well as the tooth crowns and/or part of the oral mucosa. Statistical shape models are used, for example, to describe anatomical shapes, here for example the tooth axes, and modelled in particular the probability of the presence of certain shapes. In a third step, a representation of the received digital 3D surface visualization is generated by defining a plurality of landmarks on at least one dental crown and/or parts of the oral mucosa that are visible in the digital 3D surface visualization provided. These may be anatomical landmarks such as the dental cusps, for example. Finally, in a fourth step, a representation of the tooth axes of one or more of the teeth intended for the implant-supported tooth replacement is determined based on the generated landmark-based representation of the digital 3D surface visualization and the statistical parametric 3D shape model. This procedure has the advantage that the statistical parametric 3D shape model can be used purely from the information of the digital 3D surface visualization, i.e. without additional, radiation-intensive imaging, to infer shapes that are not visible in the digital 3D surface visualization, such as the tooth axes or tooth roots. Radiation-intensive CBCT imaging is no longer necessary and anatomical structures can be accurately calculated purely on the basis of a surface scan of at least part of the existing tooth crowns and/or oral mucosa using the statistical parametric shape model.

Another aspect of the invention relates to a system for supporting a preoperative planning of a placement of a dental implant in a jaw of a patient. Such a system comprises several units. Firstly, a data providing unit configured to provide a digital 3D surface visualization. Secondly, a model providing unit configured to provide a statistical parametric 3D shape model. Thirdly, an image processing unit configured to generate a representation of the digital 3D surface visualization provided by the data providing unit by defining a plurality of landmarks located on the 3D surface visualization. Fourthly, an implant planning unit configured to determine a representation of the tooth axes of one or more of the teeth intended for the implant-supported tooth replacement. The implant planning unit does this based on the landmark-based representation of the digital 3D surface visualization generated by the image processing unit and a statistical parametric 3D shape model provided by the model providing unit.

Another aspect relates to a computer program product for supporting a preoperative planning of a placement of a dental implant in a jaw of a patient, comprising computer readable instructions for performing the methods according to the invention.

Embodiments of the invention are described below with reference to the figures.

Show in the figures:

FIG. 1 an example of a digital 3D surface visualization of a patient;

FIG. 2 an example of a CBCT imaging dataset (3D imaging dataset) of the patient;

FIG. 3 an example of an overlay of the 3D surface visualization and the 3D imaging data set of the patient;

FIG. 4 an example of a virtually inserted dental implant in the patient;

FIG. 5 an example of 2D X-ray imaging (orthopantomogram) of the patient;

FIG. 6 an example of a representation of an implant and its axis in additional imaging;

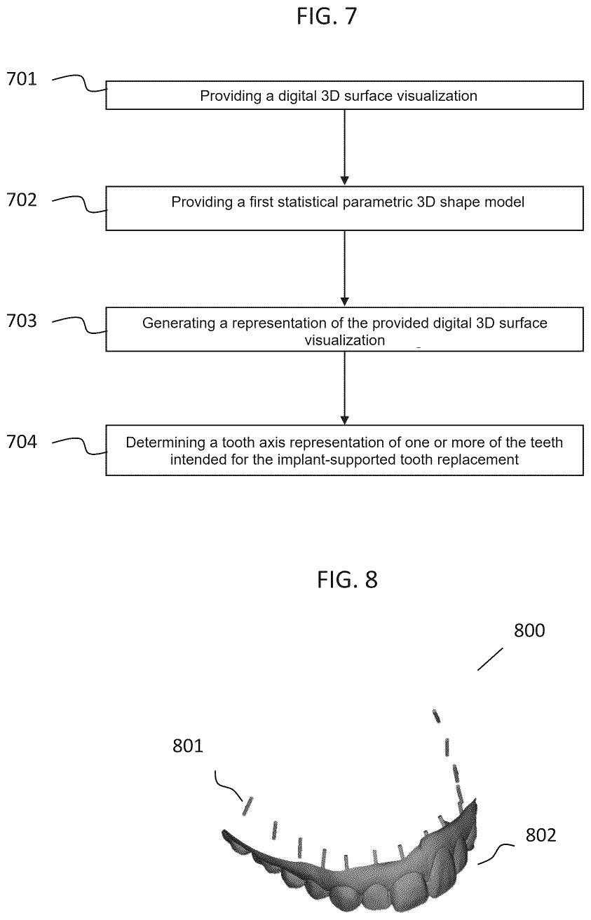

FIG. 7 an embodiment of a method for supporting a preoperative process for planning a placement of a dental implant in a jaw of a patient by means of which a tooth axis representation of one or more of the teeth intended for the implant-supported tooth replacement is determined;

FIG. 8 an example of an SSM-based reconstruction of the tooth crowns and tooth axes;

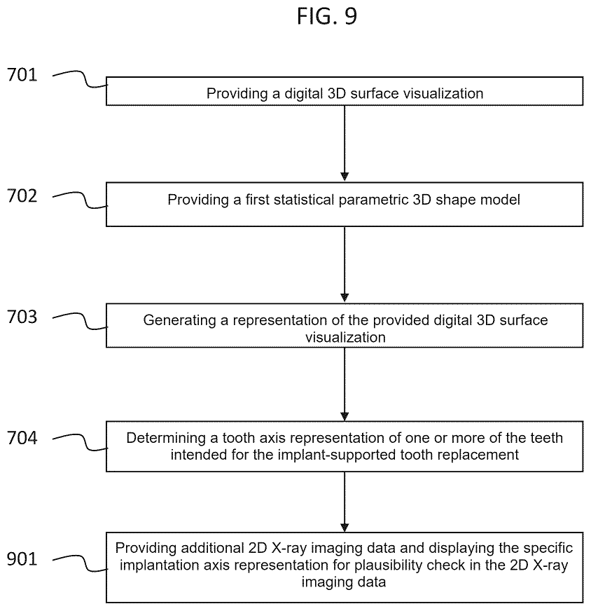

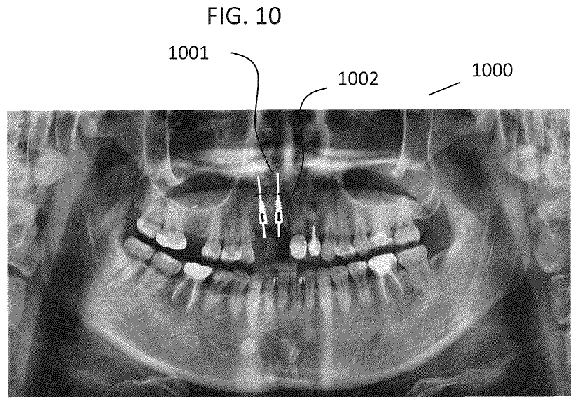

FIG. 9 an embodiment of a method for supporting a preoperative process for planning a placement of a dental implant in a jaw of a patient by means of which a tooth axis representation of one or more of the teeth intended for the implant-supported tooth replacement is determined and this is displayed in a 2D X-ray imaging set for plausibility checking;

FIG. 10 an example of a projection of an SSM-based reconstruction of the tooth axes and a representation of the implants in an orthopantomogram;

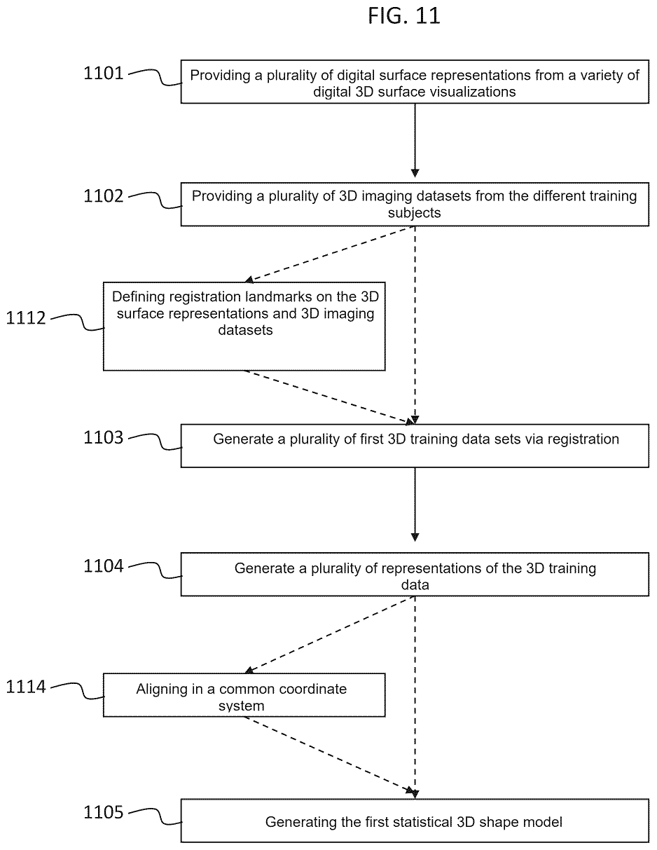

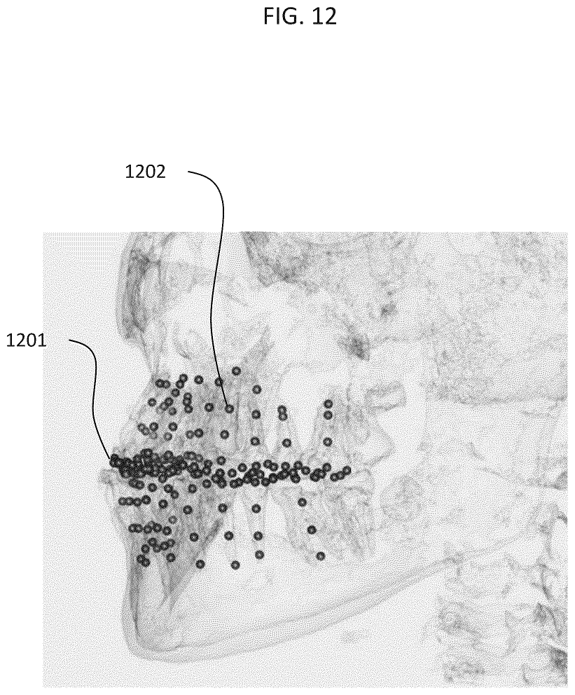

FIG. 11 an embodiment of a method for supporting a preoperative process for planning placement of a dental implant in a jaw of a patient in which a statistical parametric shape model is generated for determining tooth axes;

FIG. 12 an example of an annotation of anatomical landmarks on the surfaces of the tooth crowns and along the tooth roots;

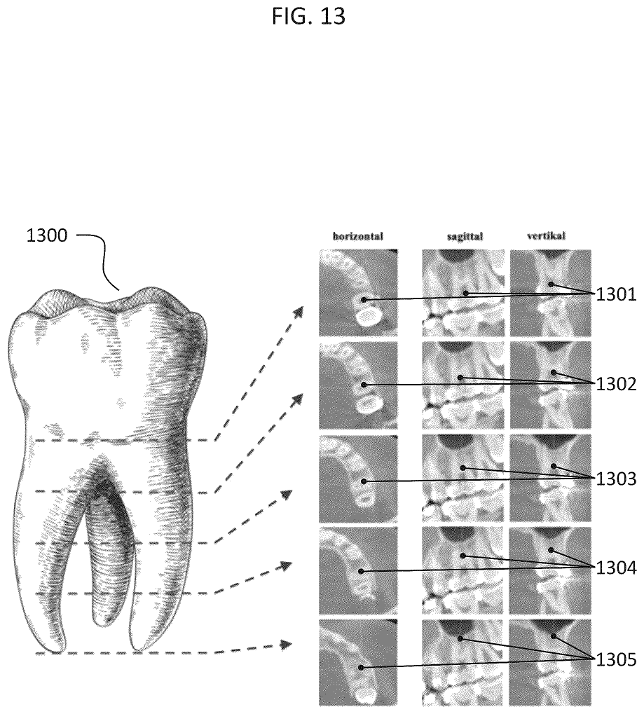

FIG. 13 shows an example of the determination of a tooth axis for a multi-rooted tooth and the annotation of corresponding landmarks;

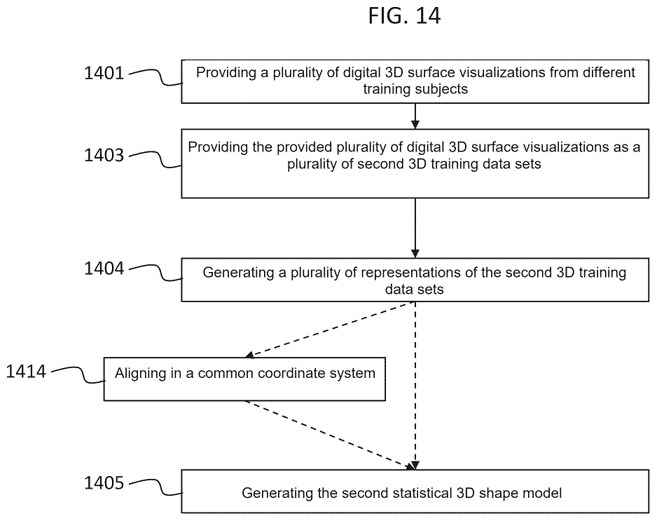

FIG. 14 an embodiment of a method for supporting a preoperative process for planning placement of a dental implant in a jaw of a patient in which a statistical parametric shape model is generated for determining dental crowns;

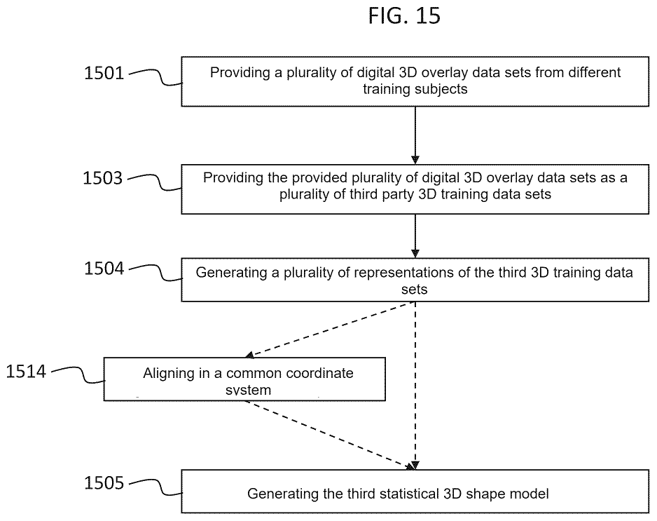

FIG. 15 an embodiment of a method for supporting a preoperative process for planning placement of a dental implant in a jaw of a patient in which a statistical parametric shape model is generated for determining implantation axes;

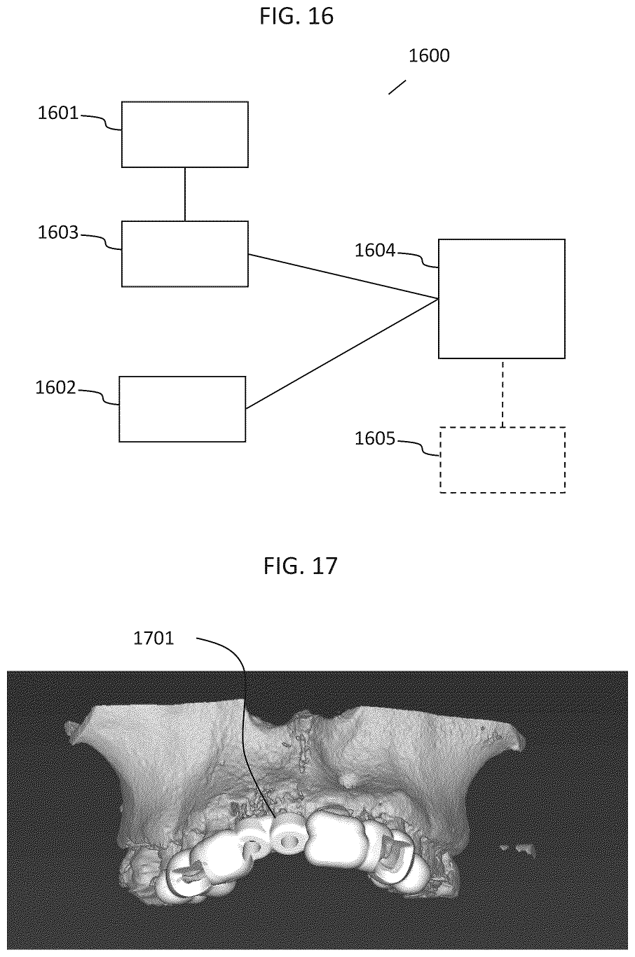

FIG. 16 an embodiment of a system for supporting a preoperative process for planning placement of a dental implant in a jaw of a patient;

FIG. 17 an example of a virtually positioned model of a drilling template.

All figures use the same reference symbols throughout for identical or similar elements. Explanations of one figure also refer analogously to the other figures.

Statistical parametric shape models (SSMs) are used in the context of the invention. SSMs are a well-validated method for shape analysis and reconstruction of data sets. They contain shape-relevant information of an object as well as its variability. Such a model estimates the average shape of an object and accordingly specifies a range within which the shape may vary. It is assumed that the shape of an object can be represented by a set of n points, whereby the points can be given in any number of dimensions, but usually in two or three dimensions. The so-called “shape” in this case is defined as the quality of the set of points that is invariant under certain transformations. In 2D or 3D, this is usually invariance under similarity transformations, i.e. rotation, translation and scaling.

An SSM therefore makes it possible to approximate shapes. This allows even incomplete shapes to be approximated to this shape using a fitting procedure of a corresponding SSM, thus generating a proposal for the most probable addition of the missing elements.

In one embodiment of the invention, the SSM used later is first generated using a plurality of training data sets. Each of these training datasets in turn consists of a plurality of points in 2, 3 or even higher dimensional space that are representative of a particular shape. A shape can also consist of several different anatomical structures, e.g. tooth crowns and roots, or even more abstract characteristics such as the tooth axes. This set of points can be generated, for example, by manual, semi-automatic or fully automatic annotation of anatomical landmarks in image data of corresponding dimensionality. Another possibility is to use the points of a surface mesh, which is used to visualize the shape, as the above-mentioned set of points. The latter can be understood in a broader sense as mathematical landmarks. In any case, the large number of training data sets consisting of points forms a point cloud. An SSM is then generated from this by modelling the distribution of the points of all training data sets. If, for example, nT shapes are available and each shape is represented by n_{L} 3-dimensional points, nr training datasets can be generated, each consisting of n_{L} 3D points. Each of these training datasets can be understood as a vector in a 3n_{L}-dimensional space. The large number of training datasets then forms a point cloud consisting of nr points in 3·n_{L}-dimensional space. The mean shape and the distribution of possible shape variations of the training data sets can then be extracted from a model of the distribution of these points.

The image data used in the context of the invention comprise 3D surface representations of the dental crowns and/or the oral mucosa (e.g. FIG. 1, 100). These can be generated, for example, by means of a laboratory scanner that produces digital 3D scans of a plaster model previously produced via a corresponding impression, or by means of an intra-oral scanner that directly scans the oral cavity. Furthermore, image data includes 3D imaging data, e.g. from a CT scanner, in particular a CBCT scanner (e.g. FIG. 2, 200), an MR scanner, an X-ray system, in particular an orthopantomograph (e.g. FIG. 5, 500) or another imaging modality. Finally, combinations of two or more of the above-mentioned image data are also possible as image data.

Landmarks are defined here as points that can be found consistently in different image data. In principle, these can be anatomical or mathematical landmarks. Anatomical landmarks refer to points that are anatomically distinguishable from other points and can therefore be found consistently; for example, cusp tips or root tips. Mathematical landmarks, on the other hand, are points that are mathematically distinguishable from other points, such as extreme points or points where there is a strong local curvature or the aforementioned points of a surface mesh. Further details on landmarks and their determination are described, for example, in section 4.1 of Cootes and Taylor, “Statistical Models of Appearance for Computer Vision Imaging Science and Biomedical Engineering” (University of Machester, 2004 published online at: https://www.face-rec.org/algorithms/aam/app_models.pdf), hereinafter referred to as COOTES.

### Use of the SSM for Reconstruction

An SSM generated as described above captures the shape variability present in the training datasets. The landmarks, as shape-representing points, encode the shape relationship between the digital 3D surface visualization and the information from the 3D imaging data. For example, shape relationships between dental crowns and/or oral mucosa and the underlying tooth roots or associated tooth axes can be modelled in this way. It is therefore possible to infer the most probable position and axis of the tooth roots or tooth axes from a digital 3D surface visualization alone using an SSM generated from the data of a sufficiently large number of training subjects. Ideally, the information from an additional, possibly radiation-intensive 3D imaging data set can be dispensed with—even if one or more teeth are missing from the digital 3D surface scans. Based solely on a plaster model or surface model 100 of a patient who is missing one or more teeth, as shown for example in FIG. 1, suggestions can be made for suitable implants and their implantation axes and/or suitable tooth crowns (tooth replacements) and possibly even other invisible anatomical structures.

FIG. 7 shows a method according to a preferred embodiment of the invention.

In step 701, digital 3D surface visualizations of at least a portion of the existing dental crowns and/or oral mucosa of a patient are provided. FIG. 1 shows an example 100 of such a 3D surface visualization. The 3D surface visualizations can be 3D scans of a plaster model using a digital scanner or image data from an intra-oral scanner. They can therefore be digital visualizations of the surfaces of the dental crowns and/or the oral mucosa, which correspond to the plaster models known in the dental field.

In step 702, a statistical parametric 3D shape model is provided, which can be used to approximate the tooth axes and optionally the tooth crowns and/or a portion of the oral mucosa. For example, representations of these structures, e.g. by landmarks or surface meshes, can be generated using the SSM. The SSM can already exist and be retrieved from a corresponding database or it can be generated initially using training data sets. The generation of a corresponding SSM is described below.

In step 703, a representation is generated from the received digital 3D surface visualization. For example, a plurality of landmarks located on the received digital 3D surface visualization are determined for this purpose. In one embodiment of the invention, all or parts of the existing tooth crowns are annotated with landmarks specified by the respective SSM to generate such a point-based representation of the digital 3D surface visualization. For example, if the corresponding teeth are also present, the same landmarks can be annotated that were also annotated during the creation of the SSM to generate the representations of the superimposed data sets. For example, landmarks can be annotated on the tooth cusps, incisal edges and/or central fissures using appropriate software. Additionally or alternatively, landmarks can be annotated on the oral mucosa. The representation(s) generated in this step can then be stored and/or transmitted in a format suitable for further processing.

In step 704, an approximation of the digital 3D surface visualization is created based on the generated representation of the digital 3D surface visualization and the SSM, i.e. a modelling based on the SSM that represents the digital 3D surface visualization as well as possible. This can be done, for example, by fitting the SSM to the patient's digital 3D surface visualization. The result is then a representation of the approximation, e.g. again in the form of anatomical or mathematical landmarks. In addition to the structures already recognizable from the digital 3D surface visualization, this representation also contains structures that are not visible in the digital 3D surface visualization but are included in the SSM. By approximation, it is therefore also possible to obtain representations of tooth roots not present in the patient data set. This approximation of originally non-existent teeth or their tooth axes can then be used as suggestions for sensible implantation axes. For example, a representation of the tooth axes of one or more of the teeth intended for the implant-supported tooth replacement can be determined using the SSM and the digital 3D surface visualization.

Using an analogue procedure, it is thus possible to generate alternative or additional suggestions for the design of the tooth replacement to be prospectively inserted. In one embodiment of the invention, a second statistical parametric 3D shape model suitable for generating representations of dental crowns is provided in step 702. Based on a representation of the digital 3D surface visualization and the second statistical parametric 3D shape model, in step 704, a representation of the dental crowns and/or a portion of the oral mucosa of one or more of the teeth intended for the implant-supported tooth replacement is also determined. The representations of the digital 3D surface representations can be generated in two different ways. If the 3D surface representations are already available as surface meshes, the points of the surface mesh are used as landmarks or points of the representation. The surface mesh thus corresponds to the representation. Another way is to annotate landmarks in the digital 3D surface visualization and use them as a representation. For example, anatomical landmarks such as dental cusps can be annotated here. Using a pure 3D surface representation, the position, shape and size of missing teeth can also be calculated on the basis of the SSM. This would replace the previous manual placement and editing of dental crowns in virtual space.

In a further embodiment of the invention, a third statistical parametric 3D shape model suitable for generating representations of implantation axes is provided in step 702. The generation of such an SSM will also be discussed below. In step 703, representations of the digital 3D surface visualizations are then generated analogously to the procedure for reconstructing the dental crowns. Based on the third statistical parametric 3D shape model and the representation of the digital 3D surface visualization generated in step 703, a representation of the implantation axes of one or more of the teeth intended for the implant-supported tooth replacement is then determined in step 704. FIG. 8 shows an example of this. In the event that the 3D surface representations are already available as surface meshes, it is no longer necessary to generate a representation and step 703 can be omitted. The generation of suggestions for implantation axes based on such empirical values is particularly advantageous because the tooth axes may deviate from the optimum implantation axes. For example, previously existing bone substance may be missing (atrophy), so that due to the lack of bone, the original tooth axes only provide a limited indication of the implantation axes to be used. By implementing existing planning data sets of implant placements in a further SSM (consisting of CBCT, 3D surface representation and defined implantation axis), not only the tooth replacement but also the implantation axis(es) to be aimed for can be calculated for each situation.

The first, second and third SSMs discussed herein may in principle also be suitable for generating several of the aforementioned representations. For example, in some embodiments, the third SSM may be suitable for generating a representation of both the tooth axes and the implantation axes, and thus suggestions for both types of axes may be generated using only a single combined SSM. Similarly, in some embodiments, an SSM may be provided for generating representations of the dental crowns and axes and possibly even the implantation axes and, based on this SSM and the provided 3D surface representation, generating representations for dental axes, crowns and implantation axes.

In either case, whether by using individual separate SSMs for tooth axes, tooth crowns and implantation axes or by using individual combined SSMs, the result may be representations for tooth axes, tooth crowns and implantation axes in a single combined representation. FIG. 8 shows such a representation 800 in which both a complete representation (e.g. as a surface mesh) of the proposed tooth axes 801 in the form of cylinders indicating direction and position and a representation of the proposed tooth crowns 802 are visible.

In a further embodiment, in addition to or as an alternative to the embodiments described above, the SSM used can also model the bone structures close to the tooth and the course of nerves close to the tooth (for example the nervus mentalis or nervus alveolaris inferior) in addition to tooth crowns, tooth roots and/or tooth axes. In turn, predictions for existing bone structures and the course of nerves could be made solely on the basis of digital surface representations, which is essential when planning and inserting an implant, e.g. to guarantee secure anchoring of the implant and to avoid damaging the surrounding nerves. The structures described must therefore be taken into account when determining an implantation axis in order to guarantee long-term success. In such an embodiment of the invention, a statistical parametric 3D shape model is thus provided in step 702, which is also suitable for generating representations of bone structures close to the teeth and/or the course of the nerves close to the teeth. In step 704, a representation of the bone structures near the teeth and/or the course of the nerves near the teeth is then additionally determined based on this statistical parametric 3D shape model and the generated representation of the digital 3D surface visualization.

In a further embodiment, in addition or as an alternative to annotating anatomical landmarks on the dental crowns, landmarks on a patient's oral mucosa may also be annotated in step 703. This may be necessary, for example, because all or most of the teeth are missing. As the bone lies close under the mucosa in the jaw area, there is a correlation between the optically detectable intraoral situation and the osseous situation of the jaws. The shape of the mucosa-covered jaw sections therefore allows regular conclusions to be drawn about the bone supply and the course of important anatomical structures. As each tooth has characteristic crown and root features, the position and course of tooth roots can also be deduced on the basis of a surface scan. Based on an SSM of the upper and lower jaw after implementing a large amount of training data, statements can also be made about the course and shape of the maxillary sinus floor and the nervus alveolaris inferior (inferior alveolar nerve, lower jaw nerve). If the SSM to be used describes possible shapes of the oral mucosa as well as the tooth axes and/or tooth crowns and possibly even the bone structures near the teeth and/or the course of the nerves near the teeth, suggestions for implants and superstructures/tooth replacements can be generated solely from surface models of the oral mucosa in a procedure analogous to the previous embodiments. For example, the creation and insertion of implant-supported superstructures for completely edentulous patients could be supported in this way. Thus, in one embodiment of the invention, such an SSM is provided in step 702 and a representation of the tooth axes of one or more of the teeth intended for the implant-supported tooth replacement is determined in step 704 based on the thus generated representation of the digital 3D surface visualization and the statistical parametric 3D shape model. In addition, representations of the tooth crowns, tooth axes and/or implantation axes of one or more of the teeth intended for the implant-supported tooth replacement and optionally the bone structures close to the teeth and/or the course of the nerves close to the teeth can optionally be determined in step 704 based on the representation of the digital 3D surface visualization and the statistical parametric 3D shape model generated in this way.

In some embodiments, an additional, imaging-based plausibility check of the generated suggestions may be provided. For example, the proposed tooth crowns, tooth axes and/or implantation axes as well as the estimated bone structures and/or nerve courses can be visualized in further imaging data, e.g. by projection. FIG. 9 shows such an embodiment example. There, in step 901, further imaging data of the osseous situation of parts of the patient's mouth, jaw and face relevant to the placement of the dental implant are provided and the determined representation of the dental crowns, dental axes and/or implantation axes and optionally the bone structures near the teeth and/or the course of the nerves near the teeth are displayed in these further imaging data for plausibility checking. For example, further imaging data, which e.g. make the real bone structures recognizable, can be used to check whether the implant axes lead into the bone and whether there is sufficient substance there for fixation or whether they run within soft tissue that is unsuitable for fixation. In a particularly preferred embodiment, the other imaging data is 2D X-ray imaging data, in particular OPG imaging data. The aim is to transfer the automatically generated implantation proposal, which is calculated by the SSM, to the OPG for the purpose of plausibility checking and adjustment. The osseous situation and the course of neighboring structures (e.g. tooth roots, nerve canal) are only visible in the OPG. In a preferred embodiment, a single-tooth fusion of the OPGs with the SSMs is performed for this purpose. The landmarks in the SSM serve as a reference for the automatically calculated regions in the OPG. A projection is achieved using the contours of the tooth crown in the OPG with the surface geometry of the SSM using a 2D-3D registration approach. This projection allows the axes to be transferred from the SSM to the OPG representation. If the user notices when reviewing the superimposed data that a change needs to be made to the implantation proposal, the user can shift and rotate the implantation axis, e.g. in order to maintain minimum distances to neighboring structures. In a particularly preferred embodiment, the known dimensions in the surface model are used to scale the OPG, which exhibits image-related distortion effects. This scaling of the OPG enables the measurement of metric distances in the OPG. By measuring the dimensions in the region of the implant, the practitioner can determine the desired implant length and diameter after adjusting the implantation axis. In another preferred embodiment, once the steps described above have been completed, all the changes made and the selected implant are transferred back to the 3D model. For this purpose, the two-dimensional changes are recalculated into the three-dimensional data set. As corrections can only be made in the plane of the X-ray image in the OPG, the three-dimensional representation in the surface model offers a new opportunity to view all three spatial planes. The oro-lingual axis in particular is not visible in the OPG due to the projection method. A final three-dimensional view of the generated implantation proposal can be used to determine whether the selected implant is perforated orally or vestibularly, for example, or whether a sufficient distance to the mucosa and the underlying bone has been maintained. An example of a plausibility check using an OPG image is shown in FIG. 10, in which the tooth axes 1001 and also implants 1002 are stylized in OPG imaging data 1000.

### Generation of the SSM

In some embodiments of the invention, the SSM used later is generated using training data sets. Each of these training datasets may comprise a single dataset or a fusion of different datasets. In one embodiment, the training data set comprises a digital 3D surface visualization fused with 3D CBCT image data. For this purpose, numerous existing CBCT images were overlaid with corresponding 3D surface representations. This creates a link between the optically detectable intraoral scan and the three-dimensional volume tomography. Based on the fused data sets as training data, the correlations between the surface and the underlying structures can now be represented in an SSM. In a further embodiment, each training data set consists of a fusion of 3D surface visualization 3D CBCT image data and a suitable representation of at least one implantation axis used in the respective patient. In a further embodiment, each training data set comprises only a 3D surface visualization. In these embodiments, shape models can therefore be created to calculate the tooth crowns, the course of the tooth root and the course of the nerve and bone, as well as implantation axes.

FIG. 11 shows the steps of a first such embodiment of the invention for generating a first SSM.

In step 1101, a plurality of digital 3D surface visualizations of at least a portion of the dental crowns and/or at least a portion of the oral mucosa are provided, each from different training subjects. The digital 3D surface visualizations 100 shown, for example, in FIG. 1 correspond to those already described above and can be obtained or generated in the same way. In a preferred embodiment, the training subjects are orthognathic, i.e. have a complete number of teeth and a regular toothing.

In step 1102, 3D imaging data sets are also provided from the same training subjects. The specific imaging modality is arbitrary as long as the tooth axes can be determined from the 3D imaging data sets thus generated and image registration with the digital 3D surface visualization of the corresponding training subject is possible with them. If, for example, both the course of the tooth roots and the tooth crowns are recognizable in the 3D imaging data, these are suitable for the procedure. In principle, the course of the tooth axis can be determined based on the course of the root(s) (more on this below). By means of points that can be determined in both data sets and correspond to each other, e.g. on the tooth crowns, both data sets can also be registered. In a preferred embodiment, the 3D imaging data comes from a CBCT. An example of 3-dimensional segmented CBCT imaging data 200 is shown in FIG. 2.

In step 1103, a plurality of training data sets is generated by aligning the digital 3D surface visualization and the 3D imaging data of each of the training subjects by means of image registration such that structures detectable in both image data overlap as well as possible. An example of such an overlay 300 is shown in FIG. 3. The 3D surface representation can serve as the reference image and the 3D imaging data as the object image, or vice versa. The object image is then optimally adapted to the reference image by means of equalizing transformation. The software solutions and image registration algorithms commonly used in medical image processing can be used here. The result of the image registration is then a combined or superimposed training data set for each training subject. For example, this then contains detailed information about the crown and root of the tooth.

In a preferred embodiment of the invention, corresponding registration landmarks are defined on the digital 3D surface visualization and the 3D imaging data prior to image registration in a step 1112. In a particularly preferred embodiment, these registration landmarks are annotated on the surfaces of the dental crowns. The registration step then attempts to match the corresponding landmarks as closely as possible. For example, an Iterative Closest Point (ICP) method can be used for registration using registration landmarks, which iteratively reduces the distance between the landmarks and thus the tooth surfaces, resulting in a superimposition with a minimum distance.

In step 1104, a representation is now generated for each of the generated training data sets. The representation must be suitable for reproducing the shapes to be considered sufficiently so that a statistical parametric shape model can be generated based on it. Usually, the respective representation is made by means of a certain number of shape-defining points, in particular the landmarks already mentioned. In one embodiment of the invention, for example, as shown in FIG. 12, training landmarks 1201 or 1202 are placed on certain points of the tooth crowns and/or the oral mucosa and along the tooth axes. For example, reliably recognizable positions such as tooth cusps, incisal edges and central fissures can be used here. The tooth axes can be determined, for example, using the component of the superimposed training data sets, which consists of the 3D imaging data. The courses of the tooth roots can usually be recognized from this data. The root canals, for example, can be used as landmarks. In the case of a tooth with a single root, the tooth axis can be determined using the root canal. When placing the landmarks, multi-rooted teeth are treated as if they had a single large root that runs along the center of the multiple real roots. The center can be determined, for example, by determining the center of the roots in the horizontal section running through several roots of the same tooth. This is repeated for several horizontal sections, resulting in a regression line of the centers determined in this way. This procedure is illustrated in FIG. 13, where the landmarks 1301-1305 are determined as described above using sectional images.

By annotating such training landmarks, a large number of representations are generated, with each representation representing all or part of the dental crowns and/or the oral mucosa as well as the dental axes of the respective training subject.

Finally, in step 1105, an SSM is generated from the training data sets. In a preferred embodiment, the generation of the SSM is based on the previously set training landmarks.

FIG. 14 shows the steps of a further embodiment of the invention for generating a second SSM.

The steps of this embodiment can be performed in addition to or separately from the steps discussed in connection with FIG. 11.

Step 1401 corresponds to step 1101 of FIG. 11.

In step 1403, the provided 3D surface representations are provided as training data sets for the second SSM. The 3D surface representations therefore serve directly as training data sets.

In step 1404, a plurality of point-based representations of the 3D training data sets are generated from this. This can be done in two ways.

Firstly, by providing an existing point-based representation for each 3D training data set provided. As discussed above, an existing point-based representation can be in the form of the surface mesh or its points. If the 3D surface representations are already available as surface meshes, e.g. as standard tessellation language (STL) files, these can be used directly as representations of training datasets.

Secondly, by generating a landmark-based representation for each provided 3D training data set by defining a plurality of training landmarks on tooth crowns of the respective training subject. This can be done, for example, in a manner analogous to that discussed in connection with FIG. 11.

In step 1405, the second statistical parametric 3D shape model is then generated based on the plurality of generated representations of the 3D training data sets. Using an SSM generated in this way, surface meshes of missing tooth crowns, for example, can be modelled.

In some embodiments, the SSM is supplemented by possible implantation axes in addition to the tooth crowns and tooth axes. This is advantageous as the tooth axes do not always correspond to the implantation axes to be aimed for, i.e. the axis along which the implant is inserted into the jaw. Predictions or recommendations for optimal implantation axes can therefore be made based on such an SSM.

FIG. 15 shows the steps of a further embodiment of the invention for generating a third SSM. The steps of this embodiment can be performed in addition to or separately from the steps discussed in connection with FIG. 11 and FIG. 14.

In step 1501, a plurality of digital 3D overlay data sets originating from the various training subjects are provided. Each of the digital 3D overlay data sets comprises an overlay of a digital 3D surface visualization 100 and a 3D imaging data set 200 of at least a portion of the existing dental crowns and/or oral mucosa of the respective training subject. In addition, a digital 3D overlay data set comprises at least one implantation axis used in a previous placement of a dental implant in the respective training subject. Furthermore, in this embodiment (implant axis SSM) it would be possible to implement the length and diameter of the selected implant. For example, each 3D data set is an overlay of the respective 3D surface representation and the 3D imaging data set into which a 3D model of the desired implant was positioned by the practitioner, taking into account all patient-specific factors and at their own discretion. The resulting data sets can, for example, be in the form of surface meshes in which the practitioner has annotated landmarks for the implantation axes used. However, data sets consisting purely of anatomical landmarks and data sets consisting purely of surface meshes are also possible.

In step 1503, the provided plurality of digital 3D overlay data sets are provided as a plurality of 3D training data sets. The digital 3D overlay data sets thus serve directly as training data sets.

In step 1504, a plurality of landmark-based representations of the 3D training data sets are generated. This can be done in two ways.

Firstly, by providing an existing point-based representation for each provided 3D training data set. As already discussed in connection with step 1404 of FIG. 14, an existing point-based representation may be in the form of the surface mesh. Thus, if the digital 3D overlay data sets are already available as surface meshes, as mixtures of a surface mesh and a set of landmarks representing the implantation axes, e.g. landmarks at the occlusal and apical ends of the implant, or as sets of landmarks representing the respective entire digital 3D overlay data sets, these can be used directly as representations of training data sets.

Secondly, by generating a landmark-based representation for each provided 3D training data set by defining a plurality of training landmarks on tooth axes and the at least one implantation axis as well as on tooth crowns and/or parts of the oral mucosa of the respective training subject. This can be done, for example, in the same way as discussed in connection with FIG. 11.

In step 1505, the third statistical 3D shape model is then generated based on the plurality of landmark-based representations of the third 3D training data sets.

In principle, the digital 3D overlay data sets used to generate the SSM described here can also be generated in an additional step analogous to step 1103 from provided 3D surface visualizations, 3D imaging data and additionally provided position, direction and length data of already used implantation axes by appropriate superimposition. In practice, however, the data is usually already superimposed after a previous placement of a dental implant. For example, the practitioner has inserted landmarks for the implantation axes in surface meshes that represent the superimposition of 3D surface representations and 3D imaging data. Such existing point-based representations for provided third 3D training data sets may thus contain annotated landmarks representing the at least one implantation axis.

In addition to the steps discussed in connection with FIGS. 11, 12 and 13, preferred embodiments include the optional step 1114 or 1414 or 1514, in which all training data sets are aligned in a uniform coordinate system with respect to their translation, rotation and optionally also their scaling in space. For example, the training data sets can be aligned in a common coordinate system using their landmarks so that corresponding landmarks in different training data sets are as close to each other as possible. In particular, the training data sets can be aligned using known optimization methods. Linear transformations are sought that minimize the distance between corresponding landmarks of the respective training data set and an average training data set. This average training dataset can be a specific reference training dataset or the average value of the already aligned training datasets. For example, an ICP algorithm can be used here to eliminate shape-irrelevant information such as position, size and rotation of this training data and to bring all training data sets into one coordinate system. As already mentioned, the scaling of the data sets, e.g. to a normalized size, can also be adjusted as an option. In a particularly preferred embodiment of the invention, however, it is assumed that the shape of a large jaw does not correspond to the enlargement of a smaller jaw and vice versa, so that in this embodiment the scaling of the individual data sets is not changed.

To generate the respective SSMs discussed above, the distributions of the point cloud representing the large number of representations of the training data sets are modelled as explained at the beginning. In particular, their expected values and variances are determined. In a particularly preferred embodiment, a principal component analysis (PCA) is applied to the training data sets for this purpose. This calculates the principal axes of the point cloud and provides the eigenvectors and corresponding eigenvalues of the covariance of the data as a result. The eigenvectors associated with the largest eigenvalues can then be used to approximate the training data sets and generate new shapes. The PCA thus provides a linear model of the shape variability of the training data and averages the shapes of all training data. Details on generating an SSM, in particular using PCA, are described in COOTES, section 4.3, for example.

In a further embodiment, the training data also comprises imaging data on the basis of which the conchal structures near the teeth and/or courses of nerves near the teeth can be determined. These can be determined, for example, from the CBCT data received in step 1102 or step 1501 or from additionally received X-ray images. Their existing representations or representations to be generated also flow into the SSM in a manner analogous to the procedure already discussed. For example, said structures and courses are also annotated with training landmarks in step 1104 or step 1504, so that the generation of the SSM is also based on these forms.

FIG. 16 shows an example of an embodiment of a system 1600 according to the invention. Such a system comprises a data providing unit 1601 configured to provide a digital 3D surface visualization. This may be, for example, a database such as PACS or a system for generating image data. The latter can in particular be an intra-oral scanner or a laboratory scanner, i.e. a 3D scanner for scanning 3D objects and in particular plaster models. In addition, it can also be one or more imaging systems such as a CT, in particular a cone beam CT (CBCT), an MRI or an X-ray system, in particular an orthopantomograph. The data providing unit can therefore comprise individual or a plurality of such systems and databases.

Furthermore, the system 1600 comprises a model providing unit 1602 for providing an SSM. This may be a database only, but the model providing unit 1602 may also be configured to generate an SSM.

In addition, the system 1600 comprises an image processing unit 1603, which is configured to generate a representation of the digital 3D surface visualization provided by the data providing unit 1601. The image processing unit 1603 can enable manual, semi-automatic or fully automatic generation of the representation. In particular, this may be a corresponding environment for manual, semi-automatic or fully automatic annotation of image data, which is also configured to forward or export the generated representations in a suitable data format. In a preferred embodiment, this is done by annotating a large number of landmarks on the 3D surface representation. For example, these may be the anatomical landmarks discussed above. In another preferred embodiment, as discussed above, the points of a surface mesh can be used as landmarks or points of a point-based representation.

Finally, the system 1600 comprises an implant planning unit 1604, which is configured to determine a representation of the tooth axes of one or more of the teeth intended for the implant-supported tooth replacement based on the representation of the digital 3D surface visualization generated by the image processing unit 1603 and a statistical parametric 3D shape model provided by the model providing unit 1602. The implant planning unit 1604 may be configured to determine the representation in the manner already explained above, in particular by adapting/fitting the SSM provided by the model providing unit 1602 to the representation of the digital 3D surface visualization provided by the image processing unit 1603.

In a further embodiment, the image processing unit 1603 may also be configured to generate a point-based representation of the digital 3D surface visualization provided by the data providing unit by providing an already existing point-based representation of the provided digital 3D surface visualization (100). The implant planning unit 1604 is then configured to determine a representation of the dental crowns and/or a part of the oral mucosa based on the representation of the digital 3D surface visualization generated by the image processing unit 1603 and a further statistical parametric 3D shape model provided by the model providing unit 1602.

In a further embodiment, the image processing unit 1603 may also be configured to generate a point-based representation of the digital 3D surface visualization provided by the data providing unit by providing an already existing point-based representation of the provided digital 3D surface visualization (100). The implant planning unit 1604 is then configured to determine a representation of at least one implantation axis based on the representation of the digital 3D surface visualization generated by the image processing unit 1603 and a further statistical parametric 3D shape model provided by the model providing unit 1602.

In further embodiments, the implant planning unit 1604 may be configured to determine a representation of the near-tooth bone structures and/or the course of the near-tooth nerves based on the representation of the digital 3D surface visualization generated by the image processing unit 1603 and further statistical parametric 3D shape models provided by the model providing unit 1602.

In one embodiment, the model providing unit 1602 may further be configured to generate an SSM in the manner already described above. Depending on the training data, this SSM may comprise representations of the tooth crowns, tooth axes and/or implant axes as well as the bone structures near the teeth and/or the course of the nerves near the teeth.

Thus, in a preferred embodiment, the data providing unit 1601 is configured to provide a plurality of digital 3D surface visualizations of at least a part of the dental crowns and/or at least a part of the oral mucosa of the respective training subject originating from different training subjects. Furthermore, it is configured to additionally provide a plurality of 3D imaging data sets originating from the different training subjects, from which tooth axes can be determined and which can be registered with the provided digital 3D surface representations belonging to the same training subject. The image processing unit 1603 of this embodiment is configured to generate a plurality of 3D training data sets by registering the provided 3D surface representations and 3D imaging data sets associated with each of the training subjects with each other. In addition, the image processing unit 1603 generates a plurality of representations of the 3D training data by generating one representation for each of the generated 3D training data sets by defining a plurality of training landmarks on tooth axes and on tooth crowns and/or parts of the oral mucosa of the respective training subject. Finally, the model providing unit 1602 is configured to generate a statistical parametric 3D shape model based on training landmarks of the plurality of representations of the 3D training data.

In a further embodiment, the image processing unit 1603 is further configured to provide the plurality of digital 3D surface visualizations provided by the data providing unit 1601 as a plurality of 3D training data sets and to generate a plurality of landmark-based representations of the second 3D training data sets as described in connection with FIG. 14. In this embodiment, the model providing unit 1602 is configured to generate a statistical parametric 3D shape model based on training landmarks of the generated plurality of representations of the 3D training data. Representations of the dental crowns, for example, can be generated from this SSM.

In a further embodiment, the data providing unit 1601 is further configured to provide the plurality of digital 3D overlay data sets originating from the various training subjects as described in connection with FIG. 15. The image processing unit 1603 is further configured to provide the plurality of digital 3D overlay data sets provided by the data providing unit 1601 as a plurality of 3D training data sets, and to generate a plurality of landmark-based representations of the second 3D training data sets as described in connection with FIG. 15. In this embodiment, the model providing unit 1602 is configured to generate a statistical parametric 3D shape model based on training landmarks of the generated plurality of representations of the 3D training data. Representations of the implantation axes, for example, can be generated from this SSM.

In further embodiments combinable with all of the above, the image processing unit 1603 may further be configured to align the individual respective 3D training data sets in a common coordinate system prior to generating the respective statistical parametric 3D shape model. In a preferred embodiment derived therefrom, the model providing unit 1602 is configured to perform a principal component analysis of the plurality of aligned respective 3D training data sets or representations thereof to generate the respective statistical parametric 3D shape model.

In a further embodiment, the data providing unit 1601 is configured to provide additional imaging data in addition to the digital 3D surface visualizations and the 3D imaging data sets. In a preferred embodiment, this is 2D X-ray images, in particular OPG imaging data. The implant planning unit 1604 is configured to display the already determined representation of the tooth crowns, tooth axes and/or implantation axes and optionally the bone structures near the teeth and/or the course of the nerves near the teeth in the further imaging data, in particular to project them into the imaging data. An example of this is shown in FIG. 10. In particular, in some embodiments, the implant planning unit 1604 may be configured to perform the plausibility check steps already described in detail above, in particular the transfer of the implantation axes into a 2D X-ray representation and, if necessary, back into the 3D model. This allows the implantologist to perform a plausibility check. For example, further imaging data, e.g. which make the real bone structures recognizable, can be used to check whether the implant axes lead into osseous tissue and whether there is sufficient substance there for fixation or whether they run within soft tissue that is completely unsuitable for fixation.

Finally, an embodiment of the system 1600 may comprise an additional drill guide unit 1605 configured to generate a drilling template and/or control instructions for an automated drilling device based on the generated implant planning data for the insertion of the implant-supported tooth replacement. An example of a model of such a drilling template 1701 is shown in FIG. 17. This may be a unit for generating control instructions for a 3D printer and an associated 3D printer. Additionally or alternatively, it may be a unit that generates control instructions for a robot and/or a corresponding surgical robot suitable for performing the necessary drilling operations.

A computer program product for supporting a preoperative process for planning placement of a dental implant in a patient's jaw includes control instructions for the procedure described above for generating representations of the tooth axes and optionally the tooth crowns, tooth axes and/or implant axes of one or more of the teeth intended for the implant-supported tooth replacement, as well as the bone structures near the teeth and/or the course of the nerves near the teeth, based on the digital 3D surface visualization and the statistical parametric 3D shape model. In particular, some embodiments of the computer program product comprise control instructions for generating SSMs as described further above. Such a computer program product may be stored on one of the conventional non-volatile storage media.

A particularly preferred embodiment is described below. If not already available, an SSM is first generated using digitized plaster models or intraoral scans and CBCT scans of various training subjects.

The plaster models are fabricated using commercially available materials, e.g. alginates, silicones or polyethers. Digitization is carried out using an optical scanner, e.g. an E3 3-shape laboratory scanner (3shape, Copenhagen, Denmark), and exported as Standard Tessellation Language (STL) files.

The CBCT scans are performed with a commercially available CBCT scanner, e.g. the 3D Accuitomo 170 CBCT scanner (Morita Corporation, Osaka, Japan); e.g. with a slice thickness of 0.25 mm and a FOV of 17×12 cm. The data sets are exported as DICOM files (Digital Imaging and Communications in Medicine) from the local image archiving and communication system.

CBCT scans can be affected by artefacts due to fixed tooth replacements or fillings, which may lead to an inadequate representation of the tooth crown. The CBCT is therefore fused with the surface scan to enable accurate visualization of the tooth crown morphology and the tooth root shape in one data set. The DICOM data sets are used to segment all osseous structures and teeth using appropriate software. In order to fuse the segmented model of the CBCT scan with the digitized plaster model, five corresponding landmarks are defined on both data sets and an iterative procedure is carried out to determine the nearest point on the lingual surface of the teeth. Specifically, the ICP method already described can be used here. The ICP method iteratively reduces the distance between the tooth surfaces, resulting in an overlay with a maximum distance of less than 0.5 mm. The result is a combined training data set containing detailed information about the tooth crown and root.

Anatomical landmarks are now used to parameterize the complex morphology of the tooth crowns and roots and to create a landmark-based SSM of the tooth shape. In order to set uniform and reproducible landmarks on the tooth crown, tooth cusps, incisal edges and central fissures are annotated. The landmark-based description of the tooth root anatomy, on the other hand, is carried out differently, as there are no clearly recognizable anatomical landmarks along tooth roots. Five landmarks are defined for each tooth root. A first landmark is placed in the center of the root canal at the level of the cementoenamel junction (CEJL). Three further landmarks are placed at a distance of two millimeters each along the tooth root in the apical direction in the center of the root canal. A fifth landmark is placed at the apical end of the root. As already described, multi-rooted teeth are treated as if they had a single large root when placing the landmarks. If multiple roots of the same tooth were cut horizontally, a landmark was placed at their center. Finally, a landmark-based model containing information about the shape of the tooth crown and root is created for each data set. FIG. 13 shows examples of the landmarks 1301-1305 of an individual in the CBCT dataset.

To determine the course of the tooth root, an idealized tooth root axis is calculated for each tooth on the basis of the landmarks 1301-1305 shown in FIG. 13, for example, using a regression line. With this method, minor errors in the subjective positioning of the root landmarks in the CBCT scan can be compensated for, as several landmarks represent the axis. Even if one landmark is incorrectly positioned, this effect is mitigated by the remaining three landmarks.

Common software packages can be used to create the SSM (e.g. the R software). All data sets contained in this training data set are aligned in terms of their translation and rotation in space in a single coordinate system. Using the landmarks described above, all available surface models are superimposed using the ICP algorithm described above. By using the ICP algorithm, information that is not relevant to the shape, such as the position and rotation of the surface scans, is eliminated and all models are brought into one coordinate system. Finally, the SSM is generated by means of a subsequent principal component analysis.

1. A computer-implemented method for supporting preoperative planning of a placement of a dental implant in a jaw of a patient, the method including steps comprising:
providing a digital 3D surface visualization of at least a portion of existing dental crowns and/or oral mucosa of the patient;
providing a first statistical parametric 3D shape model for generating representations of tooth axes;
generating a representation of the provided digital 3D surface visualization by defining a plurality of landmarks on at least one dental crown or portions of the oral mucosa visible in the provided digital 3D surface visualization; and
determining a tooth axis representation of one or more of the teeth intended for the implant-supported tooth replacement based on the generated landmark-based representation of the digital 3D surface visualization and the first statistical parametric 3D shape model.

2. The method of claim 1, further comprising:
providing a second statistical parametric 3D shape model for generating representations of dental crowns or a part of the oral mucosa;
providing a representation of the provided digital 3D surface visualization by providing a pre-existing point-based representation of the provided digital 3D surface visualization or generating a landmark-based representation of the provided digital 3D surface visualization by defining a plurality of landmarks on dental crowns or a portion of the oral mucosa of the respective training subject; and
determining a dental crown representation of the teeth intended for one or more of the implant-supported tooth replacement or a representation of a portion of the oral mucosa based on the generated representation of the digital 3D surface visualization and the second statistical parametric 3D shape model.

3. The method of claim 1, further comprising:
providing a third statistical parametric 3D shape model for generating representations of implantation axes;
providing a representation of the provided digital 3D surface visualization by providing an already existing point-based representation of the provided digital 3D surface visualization or generating a landmark-based representation of the provided digital 3D surface visualization by defining a plurality of landmarks on dental crowns or a part of the oral mucosa of the respective training subject; and
determining an implantation axis representation for one or more of the teeth intended for the implant-supported tooth replacement based on the generated representation of the provided digital 3D surface visualization and the third statistical parametric 3D shape model.

4. The method of claim 1, in which 2D X-ray imaging data of the osseous situation of parts of the patient's mouth, jaw and face relevant to the placement of the dental implant are additionally provided and the determined tooth axis representation or the determined implantation axis representation is displayed in the 2D X-ray imaging data for plausibility checking.

5. The method of claim 1, comprising generating the first statistical 3D shape model according to steps comprising:
providing a plurality of digital 3D surface visualizations, originating from different training subjects, of at least a part of the dental crowns or at least a part of the oral mucosa;
providing a plurality of 3D imaging data sets originating from the different training subjects, from which dental axes can be determined and which can each be registered with the provided digital 3D surface representations belonging to the same training subject;
generating a plurality of first 3D training data sets by registering the provided 3D surface representations and 3D imaging data sets associated with a respective one of the training subjects;
generating a plurality of representations of the first 3D training data sets by generating one representation for each generated first 3D training data set by defining a plurality of training landmarks on tooth axes and on tooth crowns or parts of the oral mucosa of the respective training subject; and
generating the first statistical parametric 3D shape model based on the training landmarks of the plurality of landmark-based representations of the first 3D training data sets.

6. The method of claim 5, wherein the registration of 3D surface representations and 3D imaging data sets belonging to a training subject is performed by matching a predetermined plurality of corresponding registration landmarks on the 3D surface representations and 3D imaging data sets to each other.

7. The method of claim 1, additionally comprising generating the second statistical 3D shape model according to steps comprising:
providing the provided plurality of digital 3D surface visualizations as a plurality of second 3D training data sets;
generating a plurality of point-based representations of the second 3D training data sets by providing a respective pre-existing point-based representation for each provided second 3D training data set or by generating a respective landmark-based representation for each provided second 3D training data set by defining a plurality of training landmarks on tooth crowns of the respective training subject; and
generating the second statistical parametric 3D shape model based on the plurality of generated representations of the second 3D training data sets.

8. The method of claim 1, additionally comprising generating the third statistical 3D shape model according to steps comprising:
providing plurality of digital 3D overlay data sets originating from the different training subjects, each of the digital 3D overlay data sets comprising an overlay of a digital 3D surface visualization of at least a part of the existing dental crowns or oral mucosa of the respective training subject and an associated 3D imaging data set as well as at least one implantation axis used in a previous placement of a dental implant in the respective training subject;
providing the provided plurality of digital 3D overlay data sets as a plurality of third 3D training data sets;
generating plurality of point-based representations of the third 3D training datasets by providing a pre-existing point-based representation for each provided third 3D training dataset or by generating a landmark-based representation for each provided third 3D training dataset by applying a plurality of training landmarks on tooth axes and the at least one implantation axis as well as on tooth crowns or parts of the oral mucosa of the respective training subject; and
generating the third statistical 3D shape model based on the plurality of generated representations of the third 3D training data sets.

9. The method of claim 8, wherein the already existing point-based representation for each provided third 3D training data set comprises annotated landmarks representing the at least one implantation axis.

10. The method of claim 5, comprising aligning the individual 3D training data sets or representations thereof in a common coordinate system prior to generating the statistical parametric 3D shape model.

11. The method of claim 10, wherein generating the statistical parametric 3D shape model comprises performing a principal component analysis of the plurality of respective 3D training data sets or representations thereof aligned with each other in a common coordinate system.

12. A system for supporting preoperative planning of placement of a dental implant in a jaw of a patient comprising:
a data providing unit configured to provide a digital 3D surface visualization a model providing unit configured to provide a statistical parametric 3D shape model;
an image processing unit configured to generate a representation of the digital 3D surface visualization provided by the data providing unit by defining a plurality of landmarks located on the 3D surface visualization;
an implant planning unit configured to determine a tooth axis representation for one or more of the teeth intended for the implant-supported tooth replacement based on the landmark-based representation of the digital 3D surface visualization generated by the image processing unit and a statistical parametric 3D shape model provided by the model providing unit.

13. The system of claim 12, wherein:
the image processing unit is further configured to generate a point-based representation of the digital 3D surface visualization provided by the data providing unit by providing an already existing point-based representation of the provided digital 3D surface visualization; and
the implant planning unit is further configured to determine a tooth crown representation of one or more of the teeth intended for the implant-supported tooth replacement or a representation of a part of the oral mucosa based on the representation of the digital 3D surface visualization generated by the image processing unit and a statistical parametric 3D shape model provided by the model providing unit.

14. The system of claim 12, wherein:
the image processing unit is further configured to generate a point-based representation of the digital 3D surface visualization provided by the data providing unit by providing an already existing point-based representation of the provided digital 3D surface visualization; and
the implant planning unit is further configured to determine a representation of the implantation axes of one or more of the teeth intended for the implant-supported tooth replacement based on the representation of the digital 3D surface visualization generated by the image processing unit and a statistical parametric 3D shape model provided by the model providing unit.

15. The system of claim 12, wherein:
the data providing unit is further configured to additionally provide 2D X-ray imaging data of the osseous situation of parts of the patient's mouth, jaw and face relevant for the placement of the dental implant; and
the implant planning unit is further configured to display the determined tooth axis representation or implant axis reconstruction in the further imaging data for plausibility checking.

16. The system of claim 12, which is configured to generate a first statistical parametric 3D shape model, in that the data providing unit is further configured to provide:
a plurality of digital 3D surface visualizations of at least a part of the dental crowns or at least a part of the oral mucosa of the respective training subject originating from different training subjects;
a plurality of 3D imaging data sets originating from the different training subjects, from which dental axes can be determined and which can each be registered with the provided digital 3D surface representations belonging to the same training subject;
wherein the image processing unit is further configured to generate:
a plurality of 3D training data sets by registering the provided 3D surface representations and 3D imaging data sets associated with each of the training subjects; and
a plurality of representations of the 3D training data by generating one representation for each of the generated 3D training data sets by defining a plurality of training landmarks on tooth axes and on tooth crowns or parts of the oral mucosa of the respective training subject; and
wherein the model providing unit is further configured to generate
a first statistical parametric 3D shape model based on the training landmarks of the plurality of representations of the 3D training data.

17. The system of claim 12, which is configured to generate a second statistical parametric 3D shape model, in that the image processing unit is further configured to:
provide the plurality of digital 3D surface visualizations as a plurality of 3D training data sets; and
generate a plurality of point-based representations of the plurality of provided 3D training data sets by providing a respective existing point-based representation for each provided 3D training data set or by generating a respective landmark-based representation for each provided 3D training data set by defining a plurality of training landmarks on tooth crowns of the respective training subject; and
wherein the model providing unit is further configured to generate a second statistical parametric 3D shape model based on the plurality of generated representations of the 3D training data.

18. The system claim 12, which is configured to generate a third statistical parametric 3D shape model, in that the data providing unit is further configured to:
provide a plurality of digital 3D overlay data sets originating from different training subjects, wherein each of the digital 3D overlay data sets comprises an overlay of a digital 3D surface visualization of at least a part of the existing dental crowns or the oral mucosa of the respective training subject and an associated 3D imaging data set as well as at least one implantation axis used in a previous placement of a dental implant in the respective training subject;
wherein the image processing unit is further configured to:
provide the plurality of digital 3D overlay data sets as a plurality of 3D training data sets; and
generate a plurality of point-based representations of the third 3D training data sets by providing a pre-existing point-based representation for each provided third 3D training data set or by generating a landmark-based representation for each provided third 3D training data set by applying a plurality of training landmarks on tooth axes and the at least one implantation axis as well as on tooth crowns or parts of the oral mucosa of the respective training subject; and
wherein the model providing unit is further configured to:
generate a third statistical parametric 3D shape model based on the plurality of generated representations of the 3D training data.

19. The system of claim 12, wherein the data providing unit comprises an intra-oral scanner or a laboratory scanner.

20. The system of claim 12, wherein the data providing unit comprises a magnetic resonance imaging scanner, computed tomography scanner, cone beam computed tomography scanner, an X-ray unit or an orthopantomography scanner.

21. The system of claim 12, further comprising a drill guide unit configured to generate a drilling template or control instructions for an automated drilling device based on the generated implant planning data for the insertion of the implant-supported tooth replacement.

22. A computer program product for supporting preoperative planning of a placement of a dental implant in a jaw of a patient, comprising computer readable instructions that, when executed by a computer processor, causes the processor to perform steps comprising:
providing a digital 3D surface visualization of at least a portion of existing dental crowns or oral mucosa of the patient;
providing a first statistical parametric 3D shape model for generating representations of tooth axes;
generating a representation of the provided digital 3D surface visualization by defining a plurality of landmarks on at least one dental crown or portions of the oral mucosa visible in the provided digital 3D surface visualization; and
determining a tooth axis representation of one or more of the teeth intended for the implant-supported tooth replacement based on the generated landmark-based representation of the digital 3D surface visualization and the first statistical parametric 3D shape model.
